(19) United States
(12) Patent Application Publication
(10) Pub. No.: US 2014/0193491 A1
(43) Pub. Date: Jul. 10, 2014

(54) PHARMACEUTICAL ANTIRETROVIRAL COMPOSITION

(71) Applicants: Geena Malhotra, Mumbai (IN); Shrinivas Madhukar Purandare, Mumbai (IN)

(72) Inventors: Geena Malhotra, Mumbai (IN); Shrinivas Madhukar Purandare, Mumbai (IN)

(73) Assignee: CIPLA LIMITED, Mumbai, MH (IN)

(21) Appl. No.: 14/119,144

(22) PCT Filed: May 30, 2012
(86) PCT No.: PCT/GB2012/000479

§ 371 Date: Feb. 14, 2014

(30) Foreign Application Priority Data
May 30, 2011 (IN) 1593-MUM/2011
May 2, 2012 (IN) 1370/MUM/2012

(51) Int. Cl. A61K 9/20; A61K 31/551; A61K 31/675; A61K 31/513

(52) U.S. Cl. CPC A61K 9/2086; A61K 31/513; A61K 31/551; A61K 31/675
USPC 424/464; 514/81

(57) ABSTRACT

The present invention relates to a pharmaceutical antiretroviral composition comprising (i) a nucleoside reverse-transcriptase inhibitor selected from lamivudine and emtricitabine, (ii) extended release nevirapine, and (iii) tenofovir; a process for preparing such composition and the use of such composition in medicine, particularly for the prophylaxis and/or treatment of diseases caused by retroviruses.

### CROSS-REFERENCE TO RELATED APPLICATIONS

This application is a filing under 35 U.S.C. 371 of International Application No. PCT/GB2012/000479 filed May 30, 2012, entitled “Pharmaceutical Antiretroviral Composition,” which claims priority to Indian Patent Application Nos. 1593/MUM/2011 filed May 30, 2011 and 1370/MUM/2012 filed on May 2, 2012, which applications are incorporated by reference herein in their entirety.

### FIELD OF INVENTION

The present invention relates to a pharmaceutical antiretroviral composition comprising at least one or more anti-retroviral agents, the manufacturing process thereof and use of the said composition for the prevention, treatment or prophylaxis of diseases caused by retroviruses, especially acquired immune deficiency syndrome or an HIV infection.

### BACKGROUND AND PRIOR ART

Demographically the second largest country in the world, India also has the third largest number of people living with HIV/AIDS. As per the provisional HIV estimate of 2008-09, by NACO (National AIDS Control Organization) there are an estimated 22.7 lakh people living with HIV/AIDS in India. The HIV prevalence rate in the country is 0.29 percent.

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus 111 (HTLV-111), now more commonly referred to as the human immunodeficiency virus or HIV.

Human immunodeficiency virus (HIV) is the etiological agent of Acquired Immune Deficiency Syndrome (AIDS) that has created a major health care problem not only in India but also globally.

HIV is a member of the class of viruses known as retroviruses. The retroviral genome is composed of RNA, which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell which plays a vital role in the body's immune system. HIV infection of these white blood cells depletes this white cell population. Eventually, the immune system is rendered inoperative and ineffective against various opportunistic diseases.

The current strategy for the treatment of HIV infection is Highly Active Antiretroviral Therapy (HAART). HAART normally consists of a combination of three or more antiretroviral drugs (ARV) taken together.

Currently available antiretroviral drugs for the treatment of HIV include nucleoside reverse transcriptase inhibitors (NRTI) or approved single pill combinations: zidovudine or AZT (Retrovir®), didanosine or DDI (Videx®), stavudine or D4T (Zenith®), lamivudine or 3TC (Epivir®), zalcitabine or DDC (Hivid®), abacavir succinate (Ziagen®), tenofovir disoproxil fumarate salt (Viread®), emtricitabine (Emtriva®), Combivir® (contains 3TC and AZT), Trizivir® (contains abacavir, 3TC and AZT); non-nucleoside reverse transcriptase inhibitors (NNRTI): nevirapine (Viramune®), delavirdine (Rescriptor®) and efavirenz (Sustiva®), peptidomimetic protease inhibitors or approved formulations: saquinavir (Invirase®, Fortovase®), indinavir (Crixivan®), ritonavir (Norvir®), nelfinavir (Viracept®), amprenavir (Agenerase®), atazanavir (Reyataz®), fosamprenavir (Lexiva®), Kaletra® (contains lopinavir and ritonavir), one fusion inhibitor enfuvirtide (T-20, Fuzeon®), Truvada® (contains Tenofovir and Emtricitabine) and Atripla® (contains fixed-dose triple combination of tenofovir, emtricitabine and efavirenz).

The goal of HAART therapy is to maximize viral suppression thus limiting and reversing damage to the immune system, leading to decline in opportunistic infections. The durability of response depends on various factors such as viral, drug and patient related factors.

Viral factors include the genetic barrier to resistance development, the capacity to remain latent and ongoing replication. Drug related factors include the potency, tolerability and convenience of a regimen and pharmacologic barriers to resistance as a function of concentrations achieved by these drugs. However, the most important patient related factor is adherence, but other factors such as toxicities, quality of life, and psychosocial issues also need to be addressed to ensure the success of therapy.

Adherence is critical for success of HAART. Numerous studies have documented that high level of adherence is needed to ensure maximal and durable suppression of the virus. (Paterson D L. et al. Adherence to protease inhibitor therapy and outcomes in patients with HIV infection Annals of Internal Medicine, 2000; 133:21-30).

Various factors influence adherence, one of which is use of different drug combinations, which are difficult to adhere to because of different dosage forms for administering each antiretroviral drug separately, this is particularly important in case of elderly patients or it may also be due to other factors such as food restrictions; treatment costs, difficulties in accessing care, and unavailability of drugs in remote places.

Since eradication of HIV is unlikely with currently available HAART and since the evidence for structured treatment interruption seems disappointing (Jintanat A. et al. Swiss HIV Cohort Study. Failures of 1 week on, 1 week off antiretroviral therapies in a randomized trial AIDS, 2003; 17:F33-F37), HIV therapy needs to be life-long coupled with high levels of adherence to the therapy; this is a demanding task for HIV infected patients due to various reasons like low morale, social stigma, low immunity attributed to the disease. Further, studies have shown that adherence to prescribed drugs over long treatment periods is generally poor. Non-adherence to HAART can lead to rebound in viral replication and, in presence of sub-optimal drug concentrations, rapid development of drug resistance. The development of drug resistance can be disastrous because of the complexity and cost associated with second line regimens and the potential for transmission of drug resistant virus in the community.

Thus, development of a fixed dose combination is a main step in simplifying the multi-drug combination therapy for improving patient adherence to the therapy since such non-adherence may contribute to the development of viral resistance and treatment failure. Further, the multi-drug combination therapy reduces the cost and also provides development of a fixed dose combination. Another advantage is that patients prefer taking one pill twice a day as compared to three pills twice a day. Convenience increases adherence, which ultimately leads to durable response in therapy.

Combination therapy, thus, reduces the daily doses to be taken by patients and simplifies dosing schedule thereby increasing patient compliance. Combination therapy also increases the drug efficacy. Use of combination therapy can yield an equivalent antiviral effect with reduced toxicity. Further, it may also reduce the risk of giving the wrong dose (high or low) of individual drugs since high doses can lead to development of serious adverse events, low doses can lead to suboptimal drug concentrations and development of drug resistance.

WO2008043829 discloses a method of treating HIV wherein emtricitabine, tenofovir and nevirapine are administered once a day. However, this application fails to mention whether the said combination is administered in a single dosage form or as a kit of parts.

WO04087169 discloses a composition useful for the treatment or prophylaxis of viral infections comprising nevirapine and at least one antiviral active compound such as alovudine.

WO2008154234 discloses extended release formulations of nevirapine. However, given the processing involved to formulate extended release formulation, the said application fails to mention as to how to formulate multi-active ingredients or medicaments with such extended release formulation.

It will further be appreciated by a person skilled in the art that extended release formulations are generally designed to be gradually absorbed during transit through the intestines, where the pH is high. Nevirapine, being a weak base agent, can be expected to exhibit low solubility in the intestines. For this reason, it is reasonable to expect that an extended release formulation of nevirapine might transit the GI tract and be excreted without sufficient dissolution and absorption of the nevirapine. This would make an extended release formulation unworkable.

In addition to the above challenge, and to meet the patient acceptance levels, patient adherence and high value treatment consideration prospects, specifically, to combat such dreadful disease/syndrome, a single complete package of such medicaments have still remained out of reach of the patients at large.

Hence, there exists a need to formulate a suitable pharmaceutical antiretroviral composition in a single complete package comprising a nucleoside reverse-transcriptase inhibitor selected from lamivudine and emtricitabine, (ii) extended release nevirapine, and (iii) tenofovir which would be convenient for patient administration thereby achieving patient adherence and exhibiting desirable dissolution.

Further, in spite of all the available antiretroviral formulations and various methods suggested in prior art there still have been difficulties such as incorporating a nucleoside reverse-transcriptase inhibitor selected from lamivudine and emtricitabine, (ii) extended release nevirapine, and (iii) tenofovir to provide a once or twice a day formulation which is stable and suitable for administration.

### OBJECT OF THE INVENTION

The object of the present invention is to provide a pharmaceutical antiretroviral composition which is suitable for oral administration as a single complete package i.e. a single kit form or single unit dosage form, optionally with pharmaceutically acceptable excipients.

Another object of the present invention is to provide a pharmaceutical antiretroviral composition comprising a single complete package, i.e. a single kit form or single unit dosage form optionally with pharmaceutically acceptable excipients for once or twice a day administration.

Yet another object of the present invention is to provide a pharmaceutical antiretroviral composition with ease of manufacture.

Still another object of the present invention is to provide a pharmaceutical antiretroviral composition for use in the prevention, treatment or prophylaxis of diseases caused by retroviruses, especially acquired immune deficiency syndrome or an HIV infection.

### SUMMARY OF THE INVENTION

According to one aspect of the present invention there is provided a pharmaceutical antiretroviral composition comprising (i) a nucleoside reverse-transcriptase inhibitor selected from lamivudine and emtricitabine, (ii) extended release nevirapine, and (iii) tenofovir.

According to another aspect of the present invention there is provided a pharmaceutical antiretroviral composition comprising (i) a nucleoside reverse-transcriptase inhibitor selected from lamivudine and emtricitabine, (ii) extended release nevirapine, and (iii) tenofovir, optionally with one or more pharmaceutically acceptable excipients, in a single unit dosage form.

According to another aspect of the present invention there is provided a pharmaceutical antiretroviral composition comprising (i) a nucleoside reverse-transcriptase inhibitor selected from lamivudine and emtricitabine, (ii) extended release nevirapine, and (iii) tenofovir, optionally with one or more pharmaceutically acceptable excipients, in a kit form.

According to another aspect of the present invention there is provided a process of manufacturing a pharmaceutical antiretroviral composition comprising (i) a nucleoside reverse-transcriptase inhibitor selected from lamivudine and emtricitabine, (ii) extended release nevirapine, and (iii) tenofovir, optionally with one or more pharmaceutically acceptable excipients.

According to yet another aspect of the present invention there is provided a method of preventing, treating or prophylaxis of diseases caused by retroviruses, especially acquired immune deficiency syndrome or an HIV infection, which method comprises administering a pharmaceutical antiretroviral composition comprising (i) a nucleoside reverse-transcriptase inhibitor selected from lamivudine and emtricitabine, (ii) extended release nevirapine, and (iii) tenofovir.

### DETAILED DESCRIPTION OF THE INVENTION

As discussed above, there is a need to develop and formulate a suitable pharmaceutical antiretroviral composition comprising once or twice a day formulation of emtricitabine or lamivudine, tenofovir and extended release nevirapine which would, not only, be convenient for patient administration but would also maintain patient adherence for such therapy.

The present invention thus provides a pharmaceutical antiretroviral composition comprising (i) a nucleoside reverse-transcriptase inhibitor selected from lamivudine and emtricitabine, (ii) extended release nevirapine, and (iii) tenofovir as a combined preparation, for simultaneous or separate use in the treatment of an HIV infection.

It will be well acknowledged by a person skilled in the art that the active ingredient nevirapine, may suitably be provided by incorporation in a pharmaceutically acceptable extended release system.

As used herein, “extended release nevirapine” means nevirapine formulated to provide a reduction in dosing frequency as compared to immediate-release nevirapine formulation as well as to provide an in vitro and/or in vivo drug release profile of extended duration, in particular relative to the release profile of an immediate release nevirapine formulation.

Further, the term “extended-release”, as used herein, refers to the release of an active ingredient from a pharmaceutical composition, in which the active ingredient is released over an extended period of time and/or at a particular location and is taken to encompass sustained-release, controlled-release, modified-release, prolonged-release, delayed-release, and the like.

Suitable nevirapine-containing extended release formulations may include, but are not limited to dissolution controlled release system, diffusion controlled release system, dissolution and diffusion controlled release system, ion exchange resin-drug complex, pH dependent formulation and osmotic pressure controlled system and any other release systems known to person skilled in the art.

It will be appreciated from the above, that the respective therapeutic agents of the combined preparation can be administered simultaneously, either in the same or different pharmaceutical formulations or separately. If there is separate administration, it will also be appreciated that the subsequently administered therapeutic agents should be administered to a patient within a time scale so as to achieve, or more particularly optimize, synergistic therapeutic effect of a combined preparation.

Thus, the present invention provides a pharmaceutical antiretroviral composition comprising (i) a nucleoside reverse-transcriptase inhibitor selected from lamivudine and emtricitabine, (ii) extended release nevirapine, and (iii) tenofovir for once or twice a day administration.

In one embodiment of the invention, the nucleoside reverse-transcriptase inhibitor is lamivudine. In an alternative embodiment of the invention, the nucleoside reverse-transcriptase inhibitor is emtricitabine.

The terms “Emtricitabine”, “Lamivudine”, “Tenofovir”, and “Nevirapine” and are used in broad sense to include not only, “Emtricitabine”, “Lamivudine”, “Tenofovir” and “Nevirapine” per se but also, their pharmaceutically acceptable salts, pharmaceutically acceptable solvates, pharmaceutically acceptable esters, pharmaceutically acceptable hydrates, pharmaceutically acceptable enantiomers, pharmaceutically acceptable derivatives, pharmaceutically acceptable polymorphs, pharmaceutically acceptable prodrugs, pharmaceutically acceptable complexes and the like.

Nevirapine, is chemically known as 11-cyclopropyl-5,11-dihydro-4-methyl-6H-dipyrido[3,2-b:2′,3′-e][1,4]diazepin-6-one, belongs to a category of non-nucleoside reverse transcriptase inhibitor (NNRTI) which is used to treat infection by HIV-I (human immunodeficiency virus, type 1). Nevirapine binds directly to reverse transcriptase (RT) and blocks the RNA-dependent and DNA-dependent DNA polymerase activities by causing a disruption of the enzyme's catalytic site. A preferred form of nevirapine is nevirapine free base. Particularly preferred forms of nevirapine include nevirapine anhydrate and nevirapine hemihydrate. A preferred dosage of nevirapine is from about 50 to about 500 mg.

A preferred form of tenofovir is tenofovir disoproxil fumarate. Tenofovir disoproxil fumarate is also known as PMPA. Tenofovir DF is a fumaric acid salt of bis-isopropoxycarbonyloxymethyl ester derivative of tenofovir. Tenofovir disoproxil fumarate is 9-[(R)-2-[[bis[[(isopropoxycarbonyl)oxy]methoxy]phosphinyl]methoxy]propyl]adenine fumarate (1:1). Tenofovir disoproxil fumarate requires initial diester hydrolysis for conversion to tenofovir and subsequent phosphorylations by cellular enzymes to form tenofovir diphosphate. Tenofovir diphosphate inhibits the activity of HTV reverse transcriptase by competing with the natural substrate deoxyadenosine 5′-triphosphate and, after incorporation into DNA, by DNA chain termination. Tenofovir diphosphate is a weak inhibitor of mammalian DNA polymerases alpha & beta and of mitochondrial DNA polymerase.

Tenofovir disoproxil fumarate is an analog of adefovir and is classified as a nucleotide reverse transcriptase inhibitor (NtRTI). Tenofovir DF is a competitive inhibitor of other naturally occurring nucleotides, and its ultimate biological activity is viral DNA chain termination. Tenofovir DF is a novel nucleotide analog with antiviral activity against both HIV and HBV. The mechanism of tenofovir DF is similar to that of nucleoside analogs, which interferes with reverse transcriptase and prevents translation of viral genetic material into viral DNA. Unlike the nucleoside analogs, the nucleotide reverse transcriptase inhibitors are chemically pre-activated with the presence of phosphate group. Since the phosphorylation step is not necessary, nucleotide analogs can incorporate into viral DNA chain more rapidly than nucleoside analogs. More importantly, this will bypass a viral mechanism of nucleoside resistance. A preferred dosage of tenofovir is from about 100 to about 300 mg.

Lamivudine (also known as 3TC) is a synthetic nucleoside analogue, chemically known as (2R,cis)-4-amino-1-(2-hydroxymethyl-1,3-oxathiolan-5-yl)-(1H)-pyrimidin-2-one (Epivir®). Lamivudine has proven antiviral activity against HIV and other viruses such as HBV.

Intracellularly, lamivudine is phosphorylated to its active 5′-triphosphate metabolite, lamivudine triphosphate (L-TP). The principal mode of action of L-TP is the inhibition of HIV-I reverse transcriptase (RT) via DNA chain termination after incorporation of the nucleoside analogue into viral DNA. L-TP is a weak inhibitor of mammalian DNA polymerases (alpha) and (beta), and mitochondrial DNA polymerase (gamma). Lamivudine has also been referred to as (−)-1-[(2R,5S) 2-(Hydroxymethyl)-1,3-oxathiolan-5-yl]cystosine, (Hydroxymethyl)-1,3-oxathiolan-5-yl]cystosine and it has proven antiviral activity against human immunodeficiency virus (HIV) and other viruses such as hepatitis B. A preferred form of lamivudine is lamivudine free base. A preferred dosage of lamivudine is from about 30 to about 300 mg.

Emtricitabine, is chemically known as 4-amino-5-fluoro-1-[2-(hydroxymethyl)-1,3-oxathiolan-5-yl]-pyrimidin-2-one, belongs to a category of nucleoside reverse transcriptase inhibitor (NRTI) which is used to treat infection by HIV-I. Specifically, emtricitabine inhibits HBV DNA polymerase and HIV-1 reverse transcriptase (RT) both in vivo and in vitro. Emtricitabine is anabolized to its triphosphate form which is the active moiety that inhibits the polymerase. A preferred form of emtricitabine is emtricitabine free base. A preferred dosage of emtricitabine is from about is from about 100 to about 300 mg.

According to a preferred embodiment, the present invention provides a pharmaceutical antiretroviral formulation comprising (i) a nucleoside reverse-transcriptase inhibitor selected from lamivudine and emtricitabine, (ii) extended release nevirapine, and (iii) tenofovir in a single unit dosage form, wherein nevirapine is incorporated/presented in any one of the extended release systems mentioned above.

It will be appreciated that the suitable extended release systems are well known to person skilled in the art, and are incorporated herein to be envisaged under the ambit of the invention. In a preferred embodiment, the extended release nevirapine is formulated to release nevirapine over a period of up to about 24 hours,

According to the above embodiment, the pharmaceutical antiretroviral composition of the present invention may preferably comprise nevirapine in an extended release form comprising at least one hydrophilic and/or hydrophobic polymer and/or water-swellable polymers, wherein. nevirapine may preferably be (i) coated with one or more of the aforementioned polymers; (ii) provided in a blend form with any of the aforementioned polymers; or (iii) provided as an active-polymer complex with suitable ratios of the active and at least one polymer.

Hydrophilic polymers that may be used herein are pharmaceutically acceptable polymeric materials having a sufficient number and distribution of hydrophilic substituents such as hydroxy and carboxy groups to impart hydrophilic properties to the polymer as a whole. The amount of hydrophilic polymer in the composition depends on the particular polymer selected, on the active pharmaceutical agent and on the desired extended release profile.

According to the present invention, pharmaceutically acceptable hydrophilic polymer for use in the pharmaceutical antiretroviral composition of the present invention may comprise one or more, but not limited to hydroxypropylmethylcellulose (HPMC, also known as hypromellose), hydroxypropylcellulose (HPC), methylcellulose, carmellose (carboxymethylcellulose), hydroxyethylcellulose (HEC), hydroxymethylcellulose, methylcellulose, carboxymethylcellulose, sodium carboxymethylcellulose, carboxymethylcellulose calcium, xanthan gum, sodium alginate, ammonium alginate, polyethylene oxide, potassium alginate, calcium alginate, propylene glycol alginate, alginic acid, polyvinyl alcohol, povidone, carbomer, guar gum, locust bean gum, potassium pectate, potassium pectinate, polyvinylpyrrolidone, polysaccharide, polyalkylene oxides polyalkyleneglycol, starch and derivatives and crosslinked homopolymers and copolymers of acrylic acid or mixtures thereof.

According to one aspect of the present invention, the hydrophilic polymer is included in an amount of from about 5% to about 50%, preferably from about 10% to about 35%, by weight of the composition.

According to the present invention, pharmaceutically acceptable hydrophobic polymer for use in the pharmaceutical antiretroviral composition of the present invention may comprise one or more, but not limited to, ethyl cellulose, cellulose acetate, cellulose acetate butyrate, cellulose acetate phthalate, cellulose acetate trimellitate, hydroxypropyl methylcellulose phthalate, poly(alkyl) methacrylate, and copolymers of acrylic or methacrylic acid esters, ammonio methyacrylate copolymer, methyacrylic acid copolymers, methacrylic acid-acrylic acid ethyl ester copolymer, methacrylic acid esters neutral copolymer, polyvinyl acetate, waxes, such as, beeswax, carnauba wax, microcrystalline wax, candelilla wax, spermaceti, montan wax, hydrogenated vegetable oil, lecithin, hydrogenated cottonseed oil, hydrogenated tallow, paraffin wax, shellac wax, petrolatum, ozokerite, and the like, as well as, synthetic waxes, e.g., polyethylene, and the like; fatty acids such as, stearic acid, palmitic acid, lauric acid, eleostearic acids, and the like; fatty alcohols, such as, lauryl alcohol, cetostearyl alcohol, stearyl alcohol, cetyl alcohol and myristyl alcohol; fatty acid esters, such as, glyceryl monostearate, glycerol monooleate, acetylated monoglycerides, tristearin, tripalmitin, cetyl esters wax, glyceryl palmitostearate and glyceryl behenate; vegetable oil, such as, hydrogenated castor oil; mineral oil or mixtures thereof.

According to one aspect of the present invention, the hydrophobic polymer is included in an amount of from about 5% to about 50%, preferably from about 10% to about 35%, by weight of the composition.

According to the present invention, pharmaceutically acceptable water-swellable polymer for use in the pharmaceutical antiretroviral composition of the present invention may comprise one or more, polyethylene oxide having a molecular weight of 100,000 to 8,000,000; poly(hydroxy alkyl methacrylate) having a molecular weight of from 30,000 to 5,000,000; poly (vinyl) alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a water-insoluble, water-swellable copolymer produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anhydride in the copolymer; Carbopol® carbomer which is as acidic carboxy polymer having a molecular weight of 450,000 to 4,000,000; Cyanamer® polyacrylamides; cross-linked water swellable indene-maleic anhydride polymers; Goodrich® polyacrylic acid having a molecular weight of 80,000 to 200,000; starch graft copolymers; Aqua Keeps® acrylate polymer polysaccharides composed of condensed glucose units such as diester cross-linked polyglucan, and the like; Amberlite® ion exchange resins; Explotab® sodium starch glycolate; Ac-Di-Sol® croscarmellose sodium or mixtures thereof.

As discussed above and hereinafter, the pharmaceutical antiretroviral composition of the present invention preferably comprises lamivudine or emtricitabine andtenofovir along with one or more pharmaceutically acceptable excipients to form an admixture, and nevirapine along with one or more extended release polymer and one or more pharmaceutically acceptable excipients to form another admixture, which admixtures are blended and/or layered to provide a single unit dosage form.

Suitably, the pharmaceutical antiretroviral composition according to the present invention are presented in solid dosage form, conveniently in unit dosage form, and include dosage form suitable for oral and buccal administration such as, but not limited to, tablets, capsules (filled with powders, pellets, beads, mini-tablets, pills, micro-pellets, small tablet units, MUPS, disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates) and sprinkles, however, other dosage forms such as liquid dosage form may be envisaged under the ambit of the invention.

It is further well known in the art that a tablet formulation is the preferred solid dosage form due to its greater stability, less risk of chemical interaction between different medicaments, smaller bulk, accurate dosage, and ease of production.

In another embodiment, the present invention provides a pharmaceutical antiretroviral composition comprising (i) a nucleoside reverse-transcriptase inhibitor selected from lamivudine and emtricitabine, (ii) extended release nevirapine, and (iii) tenofovir in a kit form.

Accordingly, the pharmaceutical antiretroviral composition in a kit form may comprise a separate unit dosage forms of a nucleoside reverse-transcriptase inhibitor selected from lamivudine and emtricitabine, tenofovir and extended release nevirapine.

According to the preferred embodiment, the pharmaceutical antiretroviral composition may be administered simultaneously, separately or sequentially in a single unit dosage form wherein the drugs and excipients are present in a single layer entity (such as a tablet or tablet in a capsule).

According to another preferred embodiment, the pharmaceutical antiretroviral composition may be administered as a bilayer tablet wherein each layer separately contains drug/drugs and pharmaceutically acceptable excipients which are then compressed to give a bilayer tablet.

According to yet another preferred embodiment, the pharmaceutical antiretroviral composition may be administered as a trilayer tablet wherein each layer separately contains drug/drugs and pharmaceutically acceptable excipients which are then compressed to give a trilayer tablet.

According to the present invention, the pharmaceutical antiretroviral composition of the present invention comprises a nucleoside reverse-transcriptase inhibitor selected from lamivudine and emtricitabine, tenofovir, and one or more pharmaceutically acceptable excipients to form a first admixture, and nevirapine in an extended release system with one or more pharmaceutically acceptable excipients to form a second admixture, which first and second admixtures are blended and compressed in a single layer to provide a single unit dosage form.

According to a preferred embodiment, the pharmaceutical antiretroviral composition of the present invention comprises a nucleoside reverse-transcriptase inhibitor selected from lamivudine and emtricitabine, tenofovir and one or more pharmaceutically acceptable excipients to form a first admixture, and nevirapine in an extended release system with one or more pharmaceutically acceptable excipients to form a second admixture, which first and second admixtures are blended and compressed to provide a bilayered unit dosage form.

According to another preferred embodiment, the pharmaceutical antiretroviral composition of the present invention comprises tenofovir and one or more pharmaceutically acceptable excipients to form a first admixture, and a nucleoside reverse-transcriptase inhibitor selected from lamivudine and emtricitabine, nevirapine in an extended release system and one or more pharmaceutically acceptable excipients to form a second admixture, which first and second admixtures are blended and compressed to provide a bilayered unit dosage form.

According to another preferred embodiment, the pharmaceutical antiretroviral composition of the present invention comprises a nucleoside reverse-transcriptase inhibitor selected from lamivudine and emtricitabine with one or more pharmaceutically acceptable excipients to form a first admixture, and tenofovir, nevirapine in an extended release system with one or more pharmaceutically acceptable excipients to form a second admixture, which first and second admixtures are blended and compressed to provide a bilayered unit dosage form.

According to another preferred embodiment, the pharmaceutical antiretroviral composition of the present invention comprises a nucleoside reverse-transcriptase inhibitor selected from lamivudine and emtricitabine, with one or more pharmaceutically acceptable excipients to form a first admixture, tenofovir along with one or more pharmaceutically acceptable excipients to form a second admixture and nevirapine in an extended release system with one or more pharmaceutically acceptable excipients to form a third admixture, which first, second and third admixtures are blended and compressed to provide a trilayered unit dosage form.

According to one embodiment of the invention, there is provided a process for preparing a pharmaceutical composition of the type described herein, which process comprises admixing (i) a nucleoside reverse-transcriptase inhibitor selected from lamivudine and emtricitabine, (ii) extended release nevirapine, and (iii) tenofovir with one or more pharmaceutically acceptable excipients.

The pharmaceutical antiretroviral composition, according to the present invention, may be prepared through various techniques or processes known in the art which includes, but are not limited to direct compression, wet granulation, dry granulation, melt granulation, melt extrusion, spray drying, solution evaporation or combinations thereof.

It will be acknowledged to a person skilled in the art, that the above mentioned techniques may be used either singly or in combination with other above mentioned techniques to provide a single layer, bilayer or trilayer or multilayer unit dosage form.

Suitable excipients may be used for formulating the various dosage forms according to the present invention.

According to the present invention, pharmaceutically acceptable carriers, diluents or fillers for use in the pharmaceutical antiretroviral composition of the present invention may comprise one or more, lactose (for example, spray-dried lactose, α-lactose, β-lactose), white sugar, lactitol, saccharose, sucrose, sugar compressible, sugar confectioners, glucose, calcium carbonate, calcium dihydrogen phosphate dihydrates, calcium phosphate-dibasic, calcium phosphate-tribasic, calcium sulfate, silicified microcrystalline cellulose, cellulose powdered, fructose, kaolin, sorbitol, mannitol, dextrates, dextrins, dextrose, maltodextrin, croscarmellose sodium, microcrystalline cellulose, hydroxypropylcellulose, L-hydroxypropylcellulose (low substituted), hydroxypropyl methylcellulose (HPMC), methylcellulose polymers, hydroxyethylcellulose, sodium carboxymethylcellulose, carboxymethylene, carboxymethylhydroxyethylcellulose and other cellulose derivatives, starches or modified starches (including potato starch, corn starch, maize starch and rice starch) and mixtures thereof.

According to the present invention, pharmaceutically acceptable surfactant may comprise one or more, Polysorbates, Sodium dodecyl sulfate (sodium lauryl sulfate), Lauryl dimethyl amine oxide, Docusate sodium, Cetyltrimethyl ammonium bromide (CTAB) Polyethoxylated alcohols, Polyoxyethylenesorbitan, Octoxynol, N,N-dimethyldodecylamine-N-oxide, Hexadecyltrimethylammonium bromide, Polyoxyl 10 lauryl ether, Bile salts (sodium deoxycholate, sodium cholate), Polyoxyl castor oil, Nonylphenolethoxylate, Cyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphatedalkanolamides, Alkylphenols, ethoxylated & sulphated, Ethoxylated aliphatic alcohol, polyoxyethylene surfactants, carboxylic esters Polyethylene glycol esters, Anhydrosorbitol ester & its ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt etc.

According to the present invention, glidants, anti-adherents and lubricants may also be incorporated in the pharmaceutical antiretroviral composition of the present invention, which may comprise one or more, stearic acid and pharmaceutically acceptable salts or esters thereof (for example, magnesium stearate, calcium stearate, sodium stearyl fumarate or other metallic stearate), talc, waxes (for example, microcrystalline waxes), glycerides, glyceryl behenate, light mineral oil, PEG, silica acid or a derivative or salt thereof (for example, silicates, silicon dioxide, colloidal silicon dioxide and polymers thereof, crospovidone, magnesium aluminosilicate and/or magnesium aluminometasilicate), sucrose ester of fatty acids, hydrogenated vegetable oils (for example, hydrogenated castor oil), mineral oil, stearic acid, colloidal anhydrous silica, sucrose esters of fatty acids, microcrystalline wax, yellow beeswax, white beeswax and mixtures thereof.

According to the present invention, suitable binders may also present in the pharmaceutical antiretroviral composition of the present invention, which may comprise one or more, polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, starches, corn starch, pregelatinized starch, microcrystalline celluloses (MCC), silicified MCC, microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropyl methylcelluloses or hypromelloses and mixtures thereof or any other suitable binder.

According to the present invention, suitable disintegrants may also be present in the pharmaceutical antiretroviral composition of the present invention, which may comprise one or more, but not limited tohydroxylpropyl cellulose (HPC), low density HPC, carboxymethylcellulose (CMC), sodium CMC, calcium CMC, croscarmellose sodium; starches exemplified under examples of fillers and carboxymethyl starch, hydroxylpropyl starch, modified starch, pregelatinized starch, crystalline cellulose, sodium starch glycolate; alginic acid or a salt thereof, such as sodium alginate or their equivalents and mixtures thereof.

According to the present invention, suitable coloring agents and flavoring agents may also be present in the pharmaceutical antiretroviral composition of the present invention, selected from FDA approved colors and flavors for oral use.

It would be appreciated by a person skilled in the art, that according to the present invention, the pharmaceutical antiretroviral composition may optionally have one or more coatings, which are functional or non-functional. Functional coatings include extended-release coatings and non-functional coatings include seal coatings and elegant coatings. Additional excipients such as film forming polymers, solvents, plasticizers, anti-adherents, opacifiers, colorants, pigments, antifoam agents, and polishing agents can be used in coatings.

Suitable film-forming agents include, but are not limited to, cellulose derivatives, such as, soluble alkyl- or hydroalkyl-cellulose derivatives such as methylcelluloses, hydroxymethyl celluloses, hydroxyethyl celluloses, hydroxypropyl celluloses, hydroxymethylethyl celluloses, hydroxypropyl methylcelluloses, sodium carboxymethyl celluloses, insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acids, polyvinyl alcohols, polyvinyl acetates, polyvinylpyrrolidones, polymethacrylates and derivatives thereof, chitosan and derivatives thereof, shellac and derivatives thereof, waxes, fat substances and mixtures thereof.

Suitable enteric coating materials, include, but are not limited to, cellulosic polymers like cellulose acetate phthalates, cellulose acetate trimellitates, hydroxypropyl methylcellulose phthalates, polyvinyl acetate phthalates, etc., methacrylic acid polymers and copolymers and mixtures thereof.

Some of the excipients are used as adjuvant to the coating process, including excipients such as plasticizers, opacifiers, antiadhesives, polishing agents, and the like.

Suitable plasticizers include, but are not limited to, castor oil, diacetylated monoglycerides, dibutyl sebacate, diethyl phthalate, glycerin, polyethylene glycols, propylene glycols, triacetin, triethyl citrate, and mixtures thereof.

Suitable opacifier includes, but is not limited to, titanium dioxide.

Suitable anti-adhesive, includes, but is not limited to, talc.

Suitable polishing agents includes, but is not limited to, polyethylene glycols of various molecular weights or mixtures thereof, talc, surfactants (glycerol monostearate and poloxamers), fatty alcohols (stearyl alcohol, cetyl alcohol, lauryl alcohol and myristyl alcohol) and waxes (carnauba wax, candelilla wax and white wax) and mixtures thereof.

Suitable solvents used in the processes of preparing the pharmaceutical antiretroviral composition of the present invention, include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and mixtures thereof.

According to a preferred embodiment, the pharmaceutical antiretroviral composition of the present invention is processed by wet granulation of tenofovir and emtricitabine or lamivudine wherein the diluent, the disintegrant along with the actives tenofovir and emtricitabine or lamivudine are sifted and dried. Then, binder solution is prepared by first dissolving the binder in purified water. Granulation is carried out by spraying of the binder solution to the above dry mixture of the ingredients, after which the formed granules are dried, sifted through the specified mesh. Extended release nevirapine is processed by wet granulation wherein the diluent along with extended release nevirapine is sifted and dried. Granulation is carried out by spraying purified water to the above dry mixture of the ingredients, after which the formed granules are dried, sifted through the specified mesh. After unloading, the granules of tenofovir, emtricitabine or lamivudine and extended release nevirapine were lubricated. The granules as obtained above are compressed to provide a single layered tablet or compressed separately to provide a bilayered tablet or a trilayered tablet. The tablets thus obtained via the process are then sprayed with a coating suspension made of ready colour mix system.

Alternatively, after compression into tablets, they can be further seal coated and then sprayed with a coating suspension made of ready colour mix system.

Alternatively, the pharmaceutical antiretroviral composition according to the present invention may also comprise the actives in nanosizeform. Preferably, the active pharmaceutical ingredients have average particle size less than about 2000 nm, preferably less than about 1000 nm.

Nanonization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nano-formulations in “Nanonization strategies for poorly water-soluble drugs,” Drug Discovery Today, Volume 00, Number 00, March 2010].

Nano-sizing leads to increase in the exposure of surface area of particles leading to an increase in the rate of dissolution.

The nanoparticles of the present invention can be obtained by any of the process such as but not limited to milling, precipitation and homogenization.

Accordingly, the process of milling comprises dispersing drug particles in a liquid dispersion medium in which the drug is poorly soluble, followed by applying mechanical means in the presence of grinding media to reduce the particle size of drug to the desired effective average particle size.

Accordingly, the process of precipitation involves the formation of crystallineor semi-crystalline drug nanoparticles by nucleation and the growth of drug crystals. In a typical procedure, drug molecules are first dissolved in an appropriate organic solvent such as acetone, tetrahydrofuran or N-methyl-2-pyrrolidone at a supersaturation concentration to allow for the nucleation of drug seeds. Drug nanocrystals are then formed by adding the organic mixture to an antisolvent like water in the presence of stabilizers such surfactants. The choice of solvents and stabilizers and the mixing process are key factors to control the size and stability of the drug nanocrystals.

Accordingly, the process of homogenization involves passing a suspension of crystalline drug and stabilizers through the narrow gap of a homogenizer at high pressure (500-2000 bar). The pressure creates powerful disruptive forces such as cavitation, collision and shearing, which disintegrate coarse particles to nanoparticles.

Accordingly, the process of high pressure homogenization comprises drug presuspension (containing drug in the micrometer range) by subjecting the drug to air jet milling in the presence of an aqueous surfactant solution. The presuspension is then subjected to high-pressure homogenization in which it passes through a very small homogenizer gap of ˜25 which leads to a high streaming velocity. High-pressure homogenization is based on the principle of cavitations (i.e., the formation, growth, and implosive collapse of vapor bubbles in a liquid).

Accordingly, the process of spray-freeze drying involves the atomization of an aqueous drug solution into a spray chamber filled with a cryogenic liquid (liquid nitrogen) or halocarbon refrigerant such as chlorofluorocarbon or fluorocarbon. The water is removed by sublimation after the liquid droplets solidify.

Accordingly, the process of supercritical fluid technology involves controlled crystallization of drug from dispersion in supercritical fluids, carbon dioxide.

Accordingly, the process of double emulsion/solvent evaporation technique involves preparation of oil/water (o/w) emulsions with subsequent removal of the oil phase through evaporation. The emulsions are prepared by emulsifying the organic phase containing drug, polymer and organic solvent in an aqueous solution containing emulsifier. The organic solvent diffuses out of the polymer phase and into the aqueous phase, and is then evaporated, forming drug-loaded polymeric nanoparticles.

Accordingly, the process of PRINT (Particle replication in non-wetting templates) involves utilization of a low surface energy fluoropolymeric mold that enables high-resolution imprint lithography, to fabricate a variety of organic particles. PRINT can precisely manipulate particle size of drug ranging from 20 nm to more than 100 nm.

Accordingly, the process of thermal condensation involves use of capillary aerosol generator (CAG) to produce high concentration condensation submicron to micron sized aerosols from drug solutions.

Accordingly, the process of ultrasonication involves application of ultrasound during particle synthesis or precipitation, which leads to smaller particles of drug and increased size uniformity.

Accordingly, the process of spray drying involves supplying the feed solution at room temperature and pumping it through the nozzle where it is atomized by the nozzle gas. The atomized solution is then dried by preheated drying gas in a special chamber to remove water moisture from the system, thus forming dry particles of drug.

According to a preferred embodiment of the present invention, the nano-milled drugs may be obtained by nano-milling of drugs with at least one surface stabilizer, at least one viscosity building agent and at least one polymer.

The present invention provides a pharmaceutical antiretroviral composition comprising (i) a nucleoside reverse-transcriptase inhibitor selected from lamivudine and emtricitabine, (ii) extended release nevirapine, and (iii) tenofovir for preventing, treating or prophylaxis of diseases caused by retroviruses, especially acquired immune deficiency syndrome or an HIV infection.

The present invention further provides a pharmaceutical antiretroviral composition comprising (i) a nucleoside reverse-transcriptase inhibitor selected from lamivudine and emtricitabine, (ii) extended release nevirapine, and (iii) tenofovir for simultaneous, separate or sequential for preventing, treating or prophylaxis of diseases caused by retroviruses, especially acquired immune deficiency syndrome or an HIV infection.

### EXAMPLES

The following examples are for illustrative purposes only and are not intended in any way to limit the scope of the present invention.

### Example 1

### Layer I

### Tenofovir

| | | Qty/tab |
|---|---|---|
| Sr. No | Name of Ingredients | mg |
| I | Dry Mix | |
| 1. | Tenofovir Disoproxil Fumarate | 300.0 |
| 2. | Lactose Monohydrate | 59.5 |
| 3. | Croscarmellose sodium | 20.00 |
| 4. | Corn starch | 30.00 |
| II | Binder Preparation | |
| 5. | Corn Starch | 15.00 |
| 6. | Polysorbate 80 | 3.0 |
| 7. | Purified Water | q.s. |
| III. | Lubrication | |
| 8. | Microcrystalline Cellulose | 50.0 |
| 9. | Croscarmellose sodium | 20.00 |
| 10. | Magnesium Stearate | 12.50 |
| | Total | 510.00 |

### Layer II

### Lamivudine

| | | Qty/tab |
|---|---|---|
| Sr. No | Name of Ingredients | mg |
| I | Dry Mix | |
| 1. | Lamivudine | 300.0 |
| 2. | Microcrystalline Cellulose | 33.2 |
| 3. | Sodium starch glycolate | 30.0 |
| 4. | Colour | 0.60 |
| II | Binder Preparation | |
| 5. | Corn Starch | 10.20 |
| 6. | Purified Water | q.s. |
| III. | Lubrication | |
| 7. | Sodium starch glycolate | 20.00 |
| 8. | Magnesium Stearate | 6.00 |
| | Total | 400.00 |

### Layer III

### Nevirapine

| | | Qty/tab |
|---|---|---|
| Sr. No | Name of Ingredients | mg |
| I | Dry Mix | |
| 1. | Nevirapine | 400.00 |
| 2. | Lactose Monohydrate | 200.00 |
| 3. | Colour | 0.01 |
| II | Binder Preparation | |
| 4. | Purified Water | q.s. |
| III. | Blending | |
| 5. | Hydroxypropyl methyl celluloseK4M Premium | 270.00 |
| | CR | |
| | Lubrication | |
| 6. | Magnesium Stearate | 10.00 |
| | Total | 880.00 |
| | Total of Layer I, Layer II & Layer III | 1790.00 |

### Film Coating:

| | | Qty/tab |
|---|---|---|
| Sr. No | Name of Ingredients | Mg |
| 1. | Opadry AMB OY-B 29000 Translucent INH | 18.0 |
| 2. | Purified water | q.s. |

Process:

### A) Granulation

### Preparation of Layer I

1) Premix of Tenofovir and lactose was prepared and dry mixed with croscarmellose sodium and corn starch.
2) Binder solution of corn starch and polysorbate 80 in purified water was prepared.
3) The dry mix obtained in step (1) was granulated using the binder solution prepared in step (2).
4) The granules obtained in step (3) were dried, sized and lubricated with microcrystalline cellulose, croscarmellose sodium and magnesium stearate.

### Preparation of Layer II

1) Dry mix of lamivudine, microcrystalline cellulose, Sodium starch glycolate and colour was prepared.
2) Binder solution of corn starch in purified water was prepared
3) The dry mix obtained in step (1) was granulated using the binder solution prepared in step (2).
4) The granules obtained in step (3) were dried, sized and lubricated with magnesium stearate and Sodium starch glycolate.

### Preparation of Layer III

1) Dry mix of nevirapine, colour and lactose was prepared.
2) The dry mix obtained in step (1) was granulated using water as binder.
3) The granules obtained in step (2) were dried, sized and blended with hydroxypropyl methyl cellulose K4M Premium CR and lubricated with magnesium stearate.

### B) Compression

1) Lubricated blend of Layer I, Layer II and Layer III was compressed to produce a trilayer tablet.

### C) Coating

1) Tablets so obtained were coated with Opadry solution.

### Example 2

### Layer I

### Emtricitabine & Tenofovir Disoproxil

| | | Qty/tab |
|---|---|---|
| Sr. No | Name of Ingredients | mg |
| I | Dry Mix | |
| 1. | Tenofovir Disoproxil Fumarate | 300.0 |
| 2. | Emtricitabine | 200.00 |
| 3. | Lactose Monohydrate | 50.0 |
| 4. | Croscarmellose sodium | 30.00 |
| 5. | Microcrystalline Cellulose | 200.00 |
| 6. | Pregelatinized starch | 25.00 |
| II | Binder Preparation | |
| 7. | Pregelatinized Starch | 25.00 |
| 8. | Purified Water | q.s. |
| III. | Lubrication | |
| 9. | Croscarmellose sodium | 30.00 |
| 10. | Magnesium Stearate | 10.00 |
| | Total | 850.00 |

### Layer II

### Nevirapine Extended Release

| | | Qty/tab |
|---|---|---|
| Sr. No | Name of Ingredients | mg |
| I | Dry Mix | |
| 1. | Nevirapine | 400.00 |
| 2. | Lactose Monohydrate | 200.00 |
| 3. | Hydroxy Propyl Methyl cellulose | 270.00 |
| II | Binder Preparation | |
| 4. | Purified Water | q.s. |
| III. | Lubrication | |
| 5. | Magnesium Stearate | 10.00 |
| | Total | 880.00 |
| | Total of Layer I & Layer II | 1730.00 |

### Film Coating:

| | | Qty/tab |
|---|---|---|
| Sr. No | Name of Ingredients | Mg |
| 1. | Opadry Blue II 32K 80963 INH | 25.0 |
| 2. | Purified water | q.s. |

Process:

### A) Granulation

### Preparation of Layer I

1) Tenofovir, Emtricitabine, lactose, croscarmellose, microcrystalline Cellulose, pregelatinized starch were sifted through mesh of required pore size.
2) The sifted ingredients were loaded in a Fluid bed processor and dry mixed.
3) Binder solution was prepared using pregelatinized starch and purified water.
4) Binder solution so obtained was sprayed on the mixture obtained in step 2.
5) Granules so obtained were dried, sized and lubricated.

### Preparation of Layer II

1) Nevirapine, lactose and HPMC were sifted through mesh of required pore size.
2) The sifted ingredients were loaded in a rapid mixer granulator and dry mixed.
3) Mixture obtained in step 2 was granulated using purified water
4) Granules so obtained were dried, sized and lubricated.

### B) Compression

1) Lubricated blend of Layer I and Layer II was compressed to produce a bilayer tablets.

### C) Coating

1) Tablets so obtained were coated with Opadry solution.

### Example 3

### Layer I

### Tenofovir

| | | Qty/tab |
|---|---|---|
| Sr. No | Name of Ingredients | mg |
| I | Dry Mix | |
| 1. | Tenofovir Disoproxil Fumarate | 300.0 |
| 2. | Lactose Monohydrate | 59.5 |
| 3. | Croscarmellose sodium | 20.00 |
| 4. | Corn starch | 30.00 |
| II | Binder Preparation | |
| 5. | Corn Starch | 15.00 |
| 6. | Polysorbate 80 | 3.0 |
| 7. | Purified Water | q.s. |
| III. | Lubrication | |
| 8. | Microcrystalline Cellulose | 50.0 |
| 9. | Croscarmellose sodium | 20.00 |
| 10. | Magnesium Stearate | 12.50 |
| | Total | 510.00 |

### Layer II

### Lamivudine

| | | Qty/tab |
|---|---|---|
| Sr. No | Name of Ingredients | mg |
| I | Dry Mix | |
| 1. | Lamivudine | 300.0 |
| 2. | Microcrystalline Cellulose | 33.2 |
| 3. | Sodium starch glycolate | 30.0 |
| 4. | Colour | 0.60 |
| II | Binder Preparation | |
| 5. | Corn Starch | 10.20 |
| 6. | Purified Water | q.s. |
| III. | Lubrication | |
| 7. | Sodium starch glycolate | 20.00 |
| 8. | Magnesium Stearate | 6.00 |
| | Total | 400.00 |

### Layer III

### Nevirapine Extended Release

| | | Qty/tab |
|---|---|---|
| Sr. No | Name of Ingredients | mg |
| I | Dry Mix | |
| 1. | Nevirapine | 400.00 |
| 2. | Lactose Monohydrate | 200.00 |
| 3. | Hydroxy Propyl Methyl cellulose | 270.00 |
| II | Binder Preparation | |
| 4. | Purified Water | q.s. |
| III. | Lubrication | |
| 5. | Magnesium Stearate | 10.00 |
| | Total | 880.00 |
| | Total of Layer I, Layer II and Layer III | 1790.00 |

### Film Coating:

| | | Qty/tab |
|---|---|---|
| Sr. No | Name of Ingredients | Mg |
| 1. | Opadry Blue II 32K 80963 INH | 25.0 |
| 2. | Purified water | q.s. |

Process:

### Preparation of Layer I

1) Premix of Tenofovir and lactose was prepared and dry mixed with croscarmellose sodium and corn starch.
2) Binder solution of corn starch and polysorbate 80 in purified water was prepared.
3) The dry mix obtained in step (1) was granulated using the binder solution prepared in step (2).
4) The granules obtained in step (3) were dried, sized and lubricated with microcrystalline cellulose, croscarmellose sodium and magnesium stearate.

### Preparation of Layer II

1) Dry mix of lamivudine, microcrystalline cellulose, Sodium starch glycolate and colour was prepared.
2) Binder solution of corn starch in purified water was prepared
3) The dry mix obtained in step (1) was granulated using the binder solution prepared in step (2).
4) The granules obtained in step (3) were dried, sized and lubricated with magnesium stearate and Sodium starch glycolate.

### Preparation of Layer III

1) Nevirapine, lactose and HPMC were sifted through mesh of required pore size.
2) The sifted ingredients were loaded in a rapid mixer granulator and dry mixed.
3) Mixture obtained in step 2 was granulated using purified water
4) Granules so obtained were dried, sized and lubricated.

### B) Compression

1) Lubricated blend of Layer I, Layer II and Layer III was compressed to produce a trilayer tablet.

### C) Coating

1) Tablets so obtained were coated with Opadry solution.

### Example 4

### Layer I

### Emtricitabine & Tenofovir Disoproxil

| | | Qty/tab |
|---|---|---|
| Sr. No | Name of Ingredients | mg |
| I | Dry Mix | |
| 1. | Tenofovir Disoproxil Fumarate | 300.0 |
| 2. | Emtricitabine | 200.00 |
| 3. | Lactose Monohydrate | 50.0 |
| 4. | Croscarmellose sodium | 30.00 |
| 5. | Microcrystalline Cellulose | 200.00 |
| 6. | Pregelatinized starch | 25.00 |
| II | Binder Preparation | |
| 7. | Pregelatinized Starch | 25.00 |
| 8. | Purified Water | q.s. |
| III. | Lubrication | |
| 9. | Croscarmellose sodium | 30.00 |
| 10. | Magnesium Stearate | 10.00 |
| | Total | 850.00 |

### Layer II

### Nevirapine

| | | Qty/tab |
|---|---|---|
| Sr. No | Name of Ingredients | mg |
| I | Dry Mix | |
| 1. | Nevirapine | 400.00 |
| 2. | Lactose Monohydrate | 200.00 |
| 3. | Colour | 0.01 |
| II | Binder Preparation | |
| 4. | Purified Water | q.s. |
| III. | Blending | |
| 5. | Hydroxypropyl methyl celluloseK4M Premium | 270.00 |
| | CR | |
| | Lubrication | |
| 6. | Magnesium Stearate | 10.00 |
| | Total | 880.00 |
| | Total of Layer I, Layer II & Layer III | 1790.00 |

### Film Coating:

| | | Qty/tab |
|---|---|---|
| Sr. No | Name of Ingredients | Mg |
| 1. | Opadry AMB OY-B 29000 Translucent INH | 18.0 |
| 2. | Purified water | q.s. |

Process:

### A) Granulation

### Preparation of Layer I

1) Tenofovir, Emtricitabine, lactose, croscarmellose, microcrystalline Cellulose, pregelatinized starch were sifted through mesh of required pore size.
2) The sifted ingredients were loaded in a Fluid bed processor and dry mixed.
3) Binder solution was prepared using pregelatinized starch and purified water.
4) Binder solution so obtained was sprayed on the mixture obtained in step 2.
5) Granules so obtained were dried, sized and lubricated.

### Preparation of Layer II

1) Dry mix of nevirapine, colour and lactose was prepared.
2) The dry mix obtained in step (1) was granulated using water as binder.
3) The granules obtained in step (2) were dried, sized and blended with hydroxypropyl methyl cellulose K4M Premium CR and lubricated with magnesium stearate.

### B) Compression

1) Lubricated blend of Layer I and Layer II was compressed to produce a bilayer tablets.

### C) Coating

1) Tablets so obtained were coated with Opadry solution.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A pharmaceutical antiretroviral composition comprising (i) a nucleoside reverse-transcriptase inhibitor selected from lamivudine and emtricitabine, (ii) extended release nevirapine, and (iii) tenofovir or its pharmaceutically acceptable salts, solvates, esters, hydrates, enantiomers, derivatives, polymorphs, prodrugs, complexes.

2. The pharmaceutical composition according to claim 1, wherein the nucleoside reverse-transcriptase inhibitor is lamivudine or its pharmaceutically acceptable salts, solvates, esters, hydrates, enantiomers, derivatives, polymorphs, prodrugs, complexes.

3. The pharmaceutical composition according to claim 1, wherein the nucleoside reverse-transcriptase inhibitor is emtricitabine or its pharmaceutically acceptable salts, solvates, esters, hydrates, enantiomers, derivatives, polymorphs, prodrugs, complexes.

4. The pharmaceutical composition according to claim 1 adapted for once or twice a day administration.

5. The pharmaceutical composition according to claim 1 in single complete package.

6. The pharmaceutical composition according to claim 5 in the form a single layer, or a bilayer or trilayer or multilayer tablet.

7. The pharmaceutical composition according to claim 1, wherein nevirapine is co-formulated with at least one hydrophilic and/or hydrophobic and/or water-swellable polymer.

8. The pharmaceutical composition according to claim 7, wherein nevirapine is coated or blended or complexed with one or more hydrophilic and/or hydrophobic and/or water-swellable polymers.

9. The pharmaceutical antiretroviral composition according to claim 1, comprising:
(i) a nucleoside reverse-transcriptase inhibitor selected from lamivudine and emtricitabine; tenofovir, and one or more pharmaceutically acceptable excipients to form a first admixture; and
(ii) nevirapine in an extended release system with one or more pharmaceutically acceptable excipients to form a second admixture,
which first and second admixtures are blended and compressed in a single layer to provide a single unit dosage form.

10. The pharmaceutical antiretroviral composition according to claim 1, comprising:
(i) a nucleoside reverse-transcriptase inhibitor selected from lamivudine and emtricitabine; tenofovir and one or more pharmaceutically acceptable excipients to form a first admixture; and
(ii) nevirapine in an extended release system with one or more pharmaceutically acceptable excipients to form a second admixture;
which first and second admixtures are blended and compressed to provide a bilayer unit dosage form.

11. The pharmaceutical antiretroviral composition according to claim 1, comprising:
(i) tenofovir and one or more pharmaceutically acceptable excipients to form a first admixture; and
(ii) a nucleoside reverse-transcriptase inhibitor selected from lamivudine and emtricitabine; nevirapine in an extended release system, and one or more pharmaceutically acceptable excipients to form a second admixture;
which first and second admixtures are blended and compressed to provide a bilayer unit dosage form.

12. The pharmaceutical antiretroviral composition according to claim 1, comprising:
(i) a nucleoside reverse-transcriptase inhibitor selected from lamivudine and emtricitabine with one or more pharmaceutically acceptable excipients to form a first admixture; and
(ii) tenofovir, nevirapine in an extended release system with one or more pharmaceutically acceptable excipients to form a second admixture;
which first and second admixtures are blended and compressed to provide a bilayer unit dosage form.

13. The pharmaceutical antiretroviral composition according to claim 1, comprising:
(i) a nucleoside reverse-transcriptase inhibitor selected from lamivudine and emtricitabine, with one or more pharmaceutically acceptable excipients to form a first admixture; and
(ii) tenofovir along with one or more pharmaceutically acceptable excipients to form a second admixture; and
(iii) nevirapine in an extended release system with one or more pharmaceutically acceptable excipients to form a third admixture;
which first, second and third admixtures are blended and compressed to provide a trilayer unit dosage form.

14. The pharmaceutical composition according to claim 1 as a combined preparation for simultaneous or separate use in the treatment of an HIV infection.

15. A process for preparing a pharmaceutical composition according to claim 1, which process comprises admixing (i) a nucleoside reverse-transcriptase inhibitor selected from lamivudine and emtricitabine, (ii) extended release nevirapine, and (iii) tenofovir with one or more pharmaceutically acceptable excipients.

16. A method of preventing, treating or prophylaxis of diseases caused by retroviruses, especially acquired immune deficiency syndrome or an HIV infection, which method comprises administering a pharmaceutical antiretroviral composition according to claim 1 to a patient in need thereof.

17. (canceled)
